(19) United States
(12) Patent Application Publication
(10) Pub. No.: US 2018/0028570 A1
(43) Pub. Date: Feb. 1, 2018

(54) Angiogenic Factors

(71) Applicant: UCL Business PLC, London (GB)

(72) Inventor: Richard Michael Day, London (GB)

(73) Assignee: UCL Business PLC, London (GB)

(21) Appl. No.: 15/661,437

(22) Filed: Jul. 27, 2017

Related U.S. Application Data
(60) Provisional application No. 62/369,243, filed on Aug. 1, 2016.

(51) Int. Cl. A61K 35/28; C07K 14/475; A61K 9/16; A61L 31/08; A61L 31/16; A61K 9/19; C12P 21/00; A61K 38/18

(52) U.S. Cl. CPC A61K 35/28; C12P 21/00; C07K 14/475; A61K 38/1866; A61L 31/08; A61L 31/16; A61K 9/19; A61K 9/1647; A61L 2300/414; A61L 2300/64; A61L 2300/606

(57) ABSTRACT

The disclosure relates to methods for obtaining angiogenic factors, as well as to methods of treating cardiovascular disease and methods for stimulating angiogenesis.

The disclosure relates to methods for obtaining angiogenic factors, as well as to methods of treating cardiovascular disease and methods for stimulating angiogenesis.

### BACKGROUND

Cardiovascular disease is a leading cause of death worldwide and promoting regeneration of tissue damaged by ischemia resulting from cardiovascular disease poses a significant challenge. There remains a need to deliver angiogenic factors to promote the formation of new blood vessels and thus treat ischemic tissues. Biomaterial scaffolds can be an effective way of delivering cells to a target site and surface topography is known to influence cell alignment, morphology and differentiation, and rough surfaces have been shown to affect cellular expression of biomarkers and growth factors (Ankrum & Karp. Trends Mol. Med. 2010; 16:203-209).

Mesenchymal stem cells (MSCs) have an immunomodulatory function and secrete bioactive trophic molecules. The broad spectrum of secreted factors released in response to local environmental stimuli includes growth peptides, cytokines, and chemokines and is defined as the MSC secretome (Murphy et al. Exp Mol Med. 2013; 45:e54). The secretome allows MSCs to primarily modulate the host microenvironment through paracrine actions and, as a result of this, MSCs offer therapeutic indications.

Pre-clinical studies have shown that the MSC secretome can be manipulated in vitro with physiological (hypoxic or anoxic), pharmacological (small molecule), cytokine or growth factor pre-conditioning, and genetic manipulation (Afzal et al. Antioxid. Redox Signal. 2010; 12:693-702, Kamota et al. J. Am. Coll. Cardiol. 2009; 53:1814-1822, Shi et al. Exp. Cell Res. 2009; 315:10-15, Tang et al. Mol. Cells 2009; 29:9-19 and Katare et al. Arterioscler Thromb Vasc Biol. 2013; 33:1872-80). Currently, none of these approaches is at a stage ready for clinical translation. Moreover, they are not capable of providing sustained control of the MSC secretome once the cells have been transplanted into the target tissue. Furthermore, the retention and viability of cells in the local tissue microenvironment after delivery as a dispersed suspension is often difficult to achieve, making dosing and any therapeutic outcome resulting from the MSC secretome unpredictable. There therefore remains a need not only to utilise such cells to produce angiogenic factors but to allow targeted delivery and retention of cells as well as direct interaction of their secretome with the local environment.

Thermally induced phase separation (TIPS) structures have been previously described in WO 2008/155558 and WO 2015/097464. Such scaffold structures may be produced by the application of TIPS disclosed in WO 2008/155558 and WO 2015/097464 or by any other suitable method. The teaching of WO 2008/155558 and WO 2015/097464 is hereby incorporated by reference.

The surface of TIPS microspheres is known to provide a highly effective surface topography for the rapid attachment of cells (Ahmadi et al., Acta Biomaterialia 2011; 7: 1542-1549). The present inventor has now found that forming a structure with hierarchical surface topographies, as a result of thermally induced phase separation, can increase the production of angiogenic factors from cells and provide a feasible approach for angiogenic factor delivery. In particular, it has been found that stimulation of a pro-angiogenic secretome capable of restoring blood flow in patients with cardiovascular disease, and in particular peripheral artery disease, can be achieved.

### SUMMARY

According to a first embodiment there is provided a method for obtaining an angiogenic factor from one or more cells comprising:
i) culturing a structure obtained by thermally induced phase separation, which has one or more cells which produce an angiogenic factor attached, in appropriate conditions for the one or more attached cells to produce the angiogenic factor; and
ii) isolating the angiogenic factor produced by the cells.

According to a second embodiment there is provided a method for obtaining an angiogenic factor from one or more cells comprising:
i) providing a structure obtained by thermally induced phase separation;
ii) attaching one or more cells which produce an angiogenic factor to the structure;
iii) culturing the one or more attached cells in appropriate conditions for the cells to produce the angiogenic factor; and
iv) isolating the angiogenic factor produced by the cells.

The formation of structures by TIPS, from suitable polymers, results in highly porous structures which are ideal for cell attachment and culture. The attached cells show increased secretion of angiogenic factors which can be isolated and used to treat disease, in particular, cardiovascular disease including peripheral artery disease, as well as in wound repair and the treatment of ulcers.

According to a third embodiment there is provided a method of treating cardiovascular disease comprising the administration to a human in need of such treatment, of a structure produced by thermally induced phase separation wherein the structure has one or more cells which produce an angiogenic factor attached to its surface.

According to a fourth embodiment there is provided a method of stimulating angiogenesis comprising contacting a blood vessel with a structure produced by thermally induced phase separation wherein the structure has one or more cells which produce an angiogenic factor attached to its surface.

### DETAILED DESCRIPTION

According to a first embodiment there is provided a method for obtaining an angiogenic factor from one or more cells comprising:
i) culturing a structure obtained by thermally induced phase separation, which has one or more cells which produce an angiogenic factor attached, in appropriate conditions for the one or more attached cells to produce the angiogenic factor; and
ii) isolating the angiogenic factor produced by the cells.

The TIPS structure may be produced as described in WO 2008/155558 and WO 2015/097464, hereby incorporated by reference.

The structure may be any suitable structure, including a self-supporting structure. Suitable structures include coatings and microspheres. The microspheres may be present in a bed reactor. By “coating” it is meant that a layer of polymer produced by TIPS is formed over all or part of a substrate.

The substrate may be any suitable substrate for growing cells on. For example, the substrate may be a medical device such as a stent, a microparticle, a coverslip, a microscope slide, a microfluidic device, or a cell culture plate or flask. Preferably the substrate is an implantable device or a cell culture plate or flask. Culture medium containing cells may be added to the coated substrate using techniques known to one skilled in the art in order to attach the cells to the substrate (i.e. seed the cells onto the substrate). The attachment of the cells to the TIPS structure can be controlled by controlling the density of cells seeded on the coated substrate. Cells may be seeded at any suitable density for the size of the substrate in order to allow appropriate contact with the TIPS structure. Appropriate densities will be known to one skilled in the art.

Any cell displaying anchorage properties and which produces an angiogenic factor can be attached to the treated structure. Particularly suitable anchorage dependent cells include Mesenchymal Stem Cells (MSCs), in particular Human Adipose Derived Mesenchymal Stem Cells (ADMSCs), bone marrow derived mesenchymal stem cells, induced pluripotent stem cells, embryonic stem cells, amniotic fluid and placental stem cells, mesoangioblasts, Müller stem cells, endothelial cells, endothelial progenitor cells, pericytes, CD133+ progenitor cells, CD34+ progenitor cells, smooth muscle cells, epithelial cells, mesangioblasts, myoblasts, muscle precursor cells, cardiomyocytes, myocardium cells, endocardium cells, pericardium cells, islet cells, fibroblasts, mesenchymal stromal cells and cells from the immune system such as monocytes, neutrophils, macrophages, dendritic cells, B cells and T cells.

Mesenchymal cells in particular have an immunomodulatory function and secrete bioactive trophic molecules. The broad spectrum of secreted proteins released in response to local environmental stimuli includes growth peptides, cytokines, and chemokines and is defined as the MSC secretome (Murphy et al. Exp Mol Med. 2013; 45:e54). The secretome allows MSCs to primarily modulate the host microenvironment through paracrine actions and, as a result of this, the MSC secretome offers therapeutic indications.

Preferably the cells are Human Adipose Derived Mesenchymal Stem Cells (ADMSCs).

The term angiogenic factor includes angiogenic-related factors. The angiogenic factor may be selected from Vascular Endothelial Growth Factor (VEGF), Fibroblast Growth Factor (FGF), activin A, ADAMTS-1, angiogenin, angiopoietin 1, angiopoietin 2, angiostatin/plasminogen, amphiregulin, artemin, tissue factor/factor III, CXCL16, DPPIV/CD26, EGF, EG-VEGF, endoglin/CD105, Endoglin/CD105, Endostatin/Collagen XVIII, Endothelin-1, FGF-7/KGF, GDNF, GM-CSF, HB-EGF, HGF, IGFBP-1, IGFBP-2, IGFBP-3, IL-1 beta, CXCL8/IL-8, LAP (TGF-beta 1), Leptin, CCL2/MCP-1, CCL3/MIP-1 alpha, PD-ECGF, Platelet Derived Growth Factor (PDGF-AA), PDGF-AB/PDGF-BB, Persephin, CXCL4/PF4, P/GF, Serpin B5/Maspin, Serpin E1/PAI-1, Serpin F1/PEDF, TIMP-1, TIMP-4, Thrombospondin-1, Thrombospondin-2, Transforming Growth Factor Beta (TGF-β), chemokine (C—C motif) ligand 2 (CCL2), histamine, Integrins ανβ₃, ανβ₅ and α₅β₁, VE-cadherin, CD31, ephrin, plasminogen activators, eNOS, COX-2, AC133, ID1/ID3, semaphorins and mixtures thereof. Preferably the angiogenic factor is VEGF.

Rather than a coating on a substrate, the structure may be a self-supporting structure, e.g. a microsphere. The term “microsphere” refers to one of a preparation of uniform substantially spherical particles. The term is well known in the art. Microspheres may contain a number of radial pores. This means that the pores extend from the central part of the microsphere towards the surface, preferably substantially parallel to the radii of the microsphere. The pores are preferably tubular and interconnected. The radial pores provide the microspheres with a level of mechanical strength.

The term “microsphere” as used herein may encompass a spherical particle which is of a size suitable for the attachment of cells. Preferably, the microsphere is about 10 to 2000 μm in diameter as characterised by electron microscopy, such as scanning electron microscopy. The diameter of the microsphere is preferably between 100 and 2000 μm in diameter. The pore size may also be selected depending on the diameter of the microsphere. Further, the pores are preferably regular in size, that is to say the pores are preferably substantially the same diameter, i.e., the diameter of the pores preferably differs by 10% or less. Porous microspheres have good mechanical strength due to the nature of the pores.

The structure is produced by thermally induced phase separation. In particular, the structure may be produced by any of the methods disclosed in WO 2008/155558, the disclosure of which is incorporated by reference in its entirety.

For example, when the structure is a coating on a substrate, the method of forming the structure may comprise the steps of:
i) coating the substrate with a polymer and a solvent;

ii) quenching the substrate having the polymer and solvent coating in a quenching fluid; and
iii) freeze-drying the coating to obtain the substrate coated with the structure.

The coating method may comprise a variety of methods that involve surface spraying or dipping the surface into the polymer and solvent mixture, followed by quenching in a freezing bath and freeze-drying until the solvent has solidified. The length of time taken for the solvent to solidify will depend on the properties of the solvent, the quenching solution and the thickness of the coating.

When the structure is a microsphere, the method of forming the structure may comprise the steps of:
i) dissolving a polymer in a solvent to form a solution;
ii) quenching droplets of the solution in a quenching fluid; and
iii) freeze-drying the resultant spheres.

By adjusting the polymer, solvent or ratio of polymer: solvent, or the temperature of the quenching solution, different surface features are achievable that can be tailored according to the needs. For example, a smooth surface, peppered with pores arranged in a chevron like pattern due to the solvent crystallisation is produced using neat PLGA for the TIPS process, whereas a rugged, interconnected and disrupted surface may be produced using a higher ratio of solvent to polymer or mixing water into the polymer solution. Preferably a rugged surface is produced.

Control of the porous nature of the structure is also achievable by manipulating the direction of the freeze front of the solidifying polymer structure. This can be achieved, for example, by placing the polymer coated surface onto a further cold surface with a temperature below the freezing point of the solvent.

Any suitable polymer may be used, but the polymer is preferably hydrophobic, pharmaceutically acceptable and completely soluble in a solvent. The polymer may be degradable or non-degradable. It may be synthetic or non-synthetic. A combination of polymers can be used, for example, a synthetic polymer used in combination with a non-synthetic polymer. Example polymers include poly(lactide-co-glycolide) (PLGA), poly(α-hydroxyester), polyanhydrides, polyorthoesters, polyphosphazines, polypropylene fumarate, poly(propylene-fumarate-co-ethylene glycol), polyethylene oxide, polyhydroxybutyrate (PHB), polycarbonate, polyurethane, polystyrene, and polyhydroxyvalerate (PHV). Co-polymers of two or more polymers may also be used, especially of PHB and PHV. Others include poly(α-hydroxyester)-co-PEG copolymer, or co-polymers including a pegylated drug. Natural polymers that may be used include fibrin. Preferably the polymer is not chitosan. Most preferably, the polymer is poly(lactide-co-glycolide) (PLGA).

Any appropriate solvent may be used in the production of the structure. The solvent is selected to have a higher freeze temperature higher than the temperature of the quench fluid. Example solvents include dimethylcarbonate, chloroform, acetone, dimethylchloride, tetrahydrofuran and supercritical carbon dioxide. Most preferably, the solvent is dimethylcarbonate.

Preferably the quenching solution has a freezing point below that of the solvent. The quenching fluid used to form the structure may be a liquid or a gas. Example quenching fluids include liquid nitrogen, liquid oxygen, liquid CO₂, freon, water, ethanol, methanol and mixtures thereof. Most preferably, the quenching solution is liquid nitrogen.

The attached cells are typically cultured in culture medium. The term “culture medium” encompasses the following solutions: tissue culture medium, serum, pooled platelet lysate, whole blood, saline comprising soluble protein and electrolytes, saline comprising serum, saline comprising a plasma substitute, water comprising soluble protein and electrolytes, water comprising serum, water comprising a plasma substitute, and mixtures thereof. Preferably the culture medium is selected from tissue culture medium, serum, or mixtures thereof, the culture medium being appropriate to the cell type as easily determined by one skilled in the art.

The tissue culture medium may be selected from Dulbecco's Modified Eagle's Medium (DMEM), Ham's F12, RPMI 1640, Iscove's, McCoy's, StemPro®, or mixtures thereof, including other media formulations readily apparent to those skilled in the art, including those found in Methods For Preparation of Media, Supplements and Substrate For Serum-Free Animal Cell Culture Alan R. Liss, New York (1984) and Cell & Tissue Culture: Laboratory Procedures, John Wiley & Sons Ltd., Chichester, England 1996, both of which are incorporated by reference herein in their entirety. Preferably the tissue culture medium is DMEM.

Serum is typically a complex solution of albumins, globulins, growth promoters and growth inhibitors. The serum may be obtained from a human, bovine, chicken, goat, porcine, rabbit, horse or sheep source. The serum may also be selected from autologous serum, serum substitutes, or mixtures thereof. Preferably the serum is human serum, pooled platelet lysate or Foetal Bovine Serum (FBS) or Foetal Calf Serum (FCS).

The structure maybe partially or fully submerged in the culture medium. Preferably the structures are fully submerged in the culture medium. Preferably the structures are submerged in tissue culture medium comprising from about 5% to about 95% (v/v) serum, more preferably about 5% to about 50% (v/v) serum, more preferably still from about 10% to about 20% (v/v) serum and most preferably about 10% (v/v) serum.

In order to aid culture and attachment of the cells to the TIPS structure, the TIPS structure may be treated with a solvent prior to addition of the cells to the culture medium. The solvent used to treat the TIPS structure may be selected from acetic acid, acetone, nitromethane, dioxane, tetrahydrofuran, pyridine, methyl ethyl ketone, DMSO, methyl acetate, halogenated hydrocarbons, glycerine, toluene, formamide, lower alcohols and mixtures thereof. The halogenated hydrocarbons include, but are not limited to, dichloromethane, chloroform, tetrachloroethane and trichloroethane. Lower alcohols include, but are not limited to, methanol and ethanol. Preferably the solvent is a lower alcohol, and most preferably the solvent is ethanol.

The solvent used to treat the TIPS structures may be used in any appropriate amount, for example between about 10% and about 100% (v/v). The solvent may be used in an amount between about 10% and about 90%. Preferably the solvent is used in an amount between about 70% and 100% or about 70% and 90%. The solvent is added to the culture medium comprising the structure. The solvent may be at any suitable strength or concentration apparent to one skilled in the art. The solvent may be pre-diluted in de-ionised water to a strength of about 50%, 60%, 70% or 80%.

Following addition of the cells to the surface coated with the structure, the cells are preferably incubated in order for sufficient growth to occur. Cell culture conditions are well known to one skilled in the art. Preferably, incubation occurs at about 20° C. to about 50° C., preferably at about 30° C. to about 40° C., and more preferably at about 35° C. to about 38° C. Most preferably, incubation occurs at about 37° C., optionally, in a 5% CO₂ incubator. The structures may be incubated for any suitable length of time which allows for sufficient growth of the cells. This can be determined by one skilled in the art.

Isolating the angiogenic factor can be performed by any method known to one skilled in the art. For example, the culture medium supernatant can be collected from the cell culture and the angiogenic factor isolated by conventional techniques such as centrifugation, chromatography, etc. If necessary, the angiogenic factor can additionally be released from the cells by repeated freezing and thawing of the cells, sonication, homogenisation or permeabilization of the cells by detergents and/or enzymes.

In a particularly preferred embodiment there is provided a method for obtaining VEGF from one or more ADMSCs comprising:
i) culturing a coating on a tissue culture plate or flask obtained by thermally induced phase separation, which has one or more ADMSCs attached, in appropriate conditions for the ADMSCs to produce VEGF; and
ii) isolating the VEGF produced by the cells.

A second embodiment relates to method for obtaining an angiogenic factor from one or more cells comprising:
i) providing a structure obtained by thermally induced phase separation;
ii) attaching one or more cells which produce an angiogenic factor to the structure;
iii) culturing the one or more attached cells in appropriate conditions for the cells to produce the angiogenic factor; and
iv) isolating the angiogenic factor produced by the cells.

The detailed disclosure relating to the first embodiment applies equally to the second embodiment.

In a particularly preferred embodiment there is provided a method for obtaining VEGF from one or more ADMSCs comprising:
i) providing a coating on tissue culture plate or flask by thermally induced phase separation;
ii) attaching one or more ADMSCs to the coating;
iii) culturing the ADMSCs in appropriate conditions for the cells to produce VEGF; and
iv) isolating the VEGF produced by the cells.

Preferably the first and second embodiments are carried out in vitro.

A third embodiment relates to a method of treating cardiovascular disease comprising the administration to a human in need of such treatment, of a structure produced by thermally induced phase separation wherein the structure has one or more cells which produce an angiogenic factor attached to its surface.

As described above, the present disclosure provides for increased angiogenic factor production, which can be used to stimulate blood vessel growth in a patient suffering from cardiovascular disease.

Cardiovascular disease is any disease which affects the heart or blood vessels.

Cardiovascular disease includes coronary artery diseases (CAD) such as angina and myocardial infarction (commonly known as a heart attack), stroke, hypertensive heart disease, rheumatic heart disease, cardiomyopathy, heart arrhythmia, congenital heart disease, valvular heart disease, carditis, aortic aneurysms, venous thrombosis, chronic wounds with decreased blood supply including diabetic and venous leg ulcers, and peripheral artery disease. Preferably the cardiovascular disease is peripheral artery disease.

The structure may be a microsphere or a coating on a substrate. Preferably the structure is a microsphere. Preferably, the microspheres are implantable in a human body.

The microspheres may be administered using a syringe and needle or as a paste if applied to an open wound. The microspheres loaded with cells may be premixed with an inert hydrogel to cushion them during delivery. The microspheres may be delivered into the vicinity where neovascularisation is required.

Preferably the third embodiment is carried out in vivo.

The medical device may be any instrument, apparatus, appliance, material or other article that is intended for use in a human. Such devices may be used for the prevention, treatment, or alleviation of disease or an injury, for the investigation, replacement, or modification of the anatomy or of a physiological process. Preferably the medical device is a dressing material, scaffold device, conduit, tissue culture surface or membrane, an artificial joint, heart valve, stent or a wound filler. Most preferably, the medical device is a stent.

When the substrate is a medical device, the TIPS structure may be a coating. By “coating”, it is meant that a layer of polymer produced by TIPS is formed over all or part of the exterior of the medical device. The layer of polymer may also be formed over all or part of an interior surface of the medical device.

Surface coating with the TIPS technology may be applied to devices composed of biological or synthetic materials that are substantially non-soluble in the solvent to be used. Suitable materials include decellularized matrices, metal, alloys, plastic, rubber or glass. Suitable solvents include dimethylcarbonate, chloroform, acetone, dimethylchloride, tetrahydrofuran and supercritical carbon dioxide or other solvents suitable for TIPS processing known to those skilled in the art.

Any cell displaying anchorage properties and which produces an angiogenic factor can be attached to the treated structure. Particularly suitable anchorage dependent cells include Mesenchymal Stem Cells (MSCs), in particular Human Adipose Derived Mesenchymal Stem Cells (ADMSCs), bone marrow derived mesenchymal stem cells, induced pluripotent stem cells, embryonic stem cells, amniotic fluid and placental stem cells, mesoangioblasts, Müller stem cells, endothelial cells, endothelial progenitor cells, pericytes, CD133+ progenitor cells, CD34+ progenitor cells, smooth muscle cells, epithelial cells, mesangioblasts, myoblasts, muscle precursor cells, cardiomyocytes, myocardium, endocardium, pericardium, islet cells, fibroblasts, mesenchymal stromal cells and cells from the immune system such as monocytes, neutrophils, macrophages, dendritic cells, B cells and T cells.

Preferably the cells are Human Adipose Derived Mesenchymal Stem Cells (ADMSCs).

The term angiogenic factor includes angiogenic-related factors. The angiogenic factor may be selected from Vascular Endothelial Growth Factor (VEGF), Fibroblast Growth Factor (FGF), activin A, ADAMTS-1, angiogenin, angiopoietin 1, angiopoietin 2, angiostatin/plasminogen, amphiregulin, artemin, tissue factor/factor III, CXCL16, DPPIV/CD26, EGF, EG-VEGF, endoglin/CD105, Endoglin/CD105, Endostatin/Collagen XVIII, Endothelin-1, FGF-7/KGF, GDNF, GM-CSF, HB-EGF, HGF, IGFBP-1, IGFBP-2, IGFBP-3, IL-1 beta, CXCL8/IL-8, LAP (TGF-beta 1), Leptin, CCL2/MCP-1, CCL3/MIP-1 alpha, PD-ECGF, Platelet Derived Growth Factor (PDGF-AA), PDGF-AB/PDGF-BB, Persephin, CXCL4/PF4, P/GF, Serpin B5/Maspin, Serpin E1/PAI-1, Serpin F1/PEDF, TIMP-1, TIMP-4, Thrombospondin-1, Thrombospondin-2, Transforming Growth Factor Beta (TGF-β), chemokine (C—C motif) ligand 2 (CCL2), histamine, Integrins ανβ₃, ανβ₅ and α₅β₁, VE-cadherin, CD31, ephrin, plasminogen activators, eNOS, COX-2, AC133, ID1/ID3, semaphorins and mixtures thereof. Preferably the angiogenic factor is VEGF.

The structure may additionally comprise a therapeutic agent, which may be attached to the TIPS structure or encapsulated within the structure by mixing the therapeutic agent with the polymer and solvent.

The term “therapeutic agent” includes proteins or peptides such as antibodies or functional fragments (e.g. binding fragments) thereof; nucleic acids, including oligonucleotides; monosaccharides, disaccharides, polysaccharides and derivatives thereof; carbohydrates; or active pharmaceutical ingredients (APIs). Preferably the agent is an API. The term API as used herein means a substance which can be used in a finished pharmaceutical product and is intended to furnish pharmacological activity or to otherwise have direct effect in the cure, mitigation, treatment or prevention of disease, or to have direct effect in restoring, correcting or modifying physiological functions in human beings. For example, the API may be one which is used to treat cardiovascular disease such as aspirin, statins, or vasoactive agents.

In a particularly preferred embodiment there is provided a method of treating peripheral artery disease comprising the administration to a human in need of such treatment, of a microsphere produced by thermally induced phase separation, wherein the microsphere has one or more ADMSCs attached to its surface.

A fourth embodiment relates to a method of stimulating angiogenesis comprising contacting a blood vessel with a structure produced by thermally induced phase separation wherein the structure has one or more cells which produce an angiogenic factor attached to its surface.

As described above, the present disclosure provides for increased angiogenic factor production which can stimulate angiogenesis and new blood vessel growth.

The structure may be a microsphere or a coating on a substrate as described above. Preferably the structure is microsphere. Preferably, the microspheres are implantable in a human body.

The microspheres may be administered using a syringe and needle or as a paste if applied to an open wound. The microspheres loaded with cells may be premixed with an inert hydrogel to cushion them during delivery. The microspheres may be delivered into the vicinity where neovascularisation is required.

Preferably the fourth embodiment is carried out in vivo.

When the substrate is a medical device, the TIPS structure may be a coating. By “coating”, it is meant that a layer of polymer produced by TIPS is formed over all or part of the exterior of the medical device. The layer of polymer may also be formed over all or part of an interior surface of the medical device.

Any cell displaying anchorage properties and which produces an angiogenic factor can be attached to the treated structure. Particularly suitable anchorage dependent cells include Mesenchymal Stem Cells (MSCs), in particular Human Adipose Derived Mesenchymal Stem Cells (ADMSCs), bone marrow derived mesenchymal stem cells, induced pluripotent stem cells, embryonic stem cells, amniotic fluid and placental stem cells, mesoangioblasts, Müller stem cells, endothelial cells, endothelial progenitor cells, pericytes, CD133+ progenitor cells, CD34+ progenitor cells, smooth muscle cells, epithelial cells, mesangioblasts, myoblasts, muscle precursor cells, cardiomyocytes, myocardium, endocardium, pericardium, islet cells, fibroblasts, mesenchymal stromal cells and cells from the immune system such as monocytes, neutrophils, macrophages, dendritic cells, B cells and T cells.

Preferably the cells are Human Adipose Derived Mesenchymal Stem Cells (ADMSCs).

The term angiogenic factor includes angiogenic-related factors. The angiogenic factor may be selected from Vascular Endothelial Growth Factor (VEGF), Fibroblast Growth Factor (FGF), activin A, ADAMTS-1, angiogenin, angiopoietin 1, angiopoietin 2, angiostatin/plasminogen, amphiregulin, artemin, tissue factor/factor III, CXCL16, DPPIV/CD26, EGF, EG-VEGF, endoglin/CD105, Endoglin/CD105, Endostatin/Collagen XVIII, Endothelin-1, FGF-7/KGF, GDNF, GM-CSF, HB-EGF, HGF, IGFBP-1, IGFBP-2, IGFBP-3, IL-1 beta, CXCL8/IL-8, LAP (TGF-beta 1), Leptin, CCL2/MCP-1, CCL3/MIP-1 alpha, PD-ECGF, Platelet Derived Growth Factor (PDGF-AA), PDGF-AB/PDGF-BB, Persephin, CXCL4/PF4, P/GF, Serpin B5/Maspin, Serpin E1/PAI-1, Serpin F1/PEDF, TIMP-1, TIMP-4, Thrombospondin-1, Thrombospondin-2, Transforming Growth Factor Beta (TGF-β), chemokine (C—C motif) ligand 2 (CCL2), histamine, Integrins ανβ₃, ανβ₅ and α₅β₁, VE-cadherin, CD31, ephrin, plasminogen activators, eNOS, COX-2, AC133, ID1/ID3, semaphorins and mixtures thereof. Preferably the angiogenic factor is VEGF.

The structure may additionally comprise a therapeutic agent, which may be attached to the TIPS structure or encapsulated within the structure by mixing the therapeutic agent with the polymer and solvent.

The term “therapeutic agent” includes proteins or peptides such as antibodies or functional fragments (e.g. binding fragments) thereof; nucleic acids, including oligonucleotides; monosaccharides, disaccharides, polysaccharides and derivatives thereof; carbohydrates; or active pharmaceutical ingredients (APIs). Preferably the agent is an API. The term API as used herein means a substance which can be used in a finished pharmaceutical product and is intended to furnish pharmacological activity or to otherwise have direct effect in the cure, mitigation, treatment or prevention of disease, or to have direct effect in restoring, correcting or modifying physiological functions in human beings. For example, the API may be one which is used to treat cardiovascular disease such as aspirin, statins, or vasoactive agents.

In a particularly preferred embodiment there is provided a method of stimulating angiogenesis comprising contacting a blood vessel with a microsphere produced by thermally induced phase separation, wherein the microsphere has one or more ADMSCs attached to its surface.

The embodiments will now be described in detail, by way of example only, with reference to the drawings, in which:

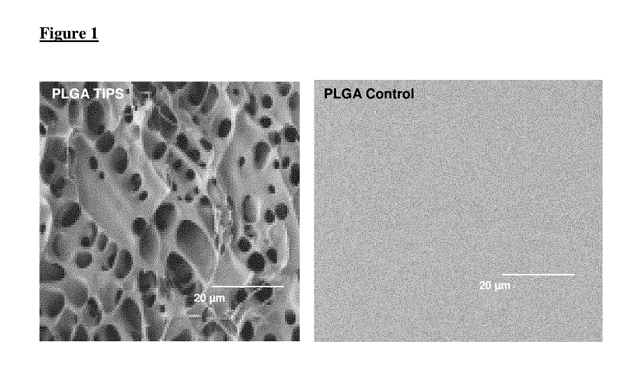

FIG. 1: Scanning Electron Micrograph (SEM) images of cell culture substrates coated with poly(DL-lactide-co-glycolide) (PLGA). The TIPS process results in a hierarchically structured porous topography compared with control surfaces coated with the same polymer.

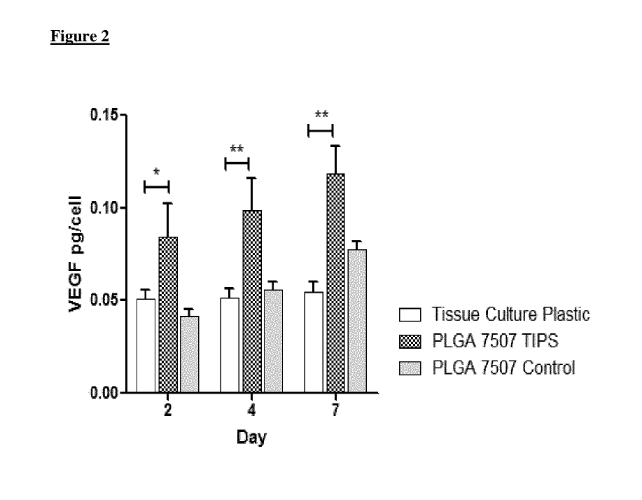

FIG. 2: Human Adipose Derived Stem Cells (ADMSCs) were cultured on TIPS or control PLGA coated surfaces and conventional polycarbonate tissue culture plastic. The supernatants were collected and the amount of VEGF secreted measured by ELISA (Enzyme-Linked Immunosorbant Assay) and normalized to the number of cells at each time point. Cumulative data are plotted from n=6 replicates for each group.

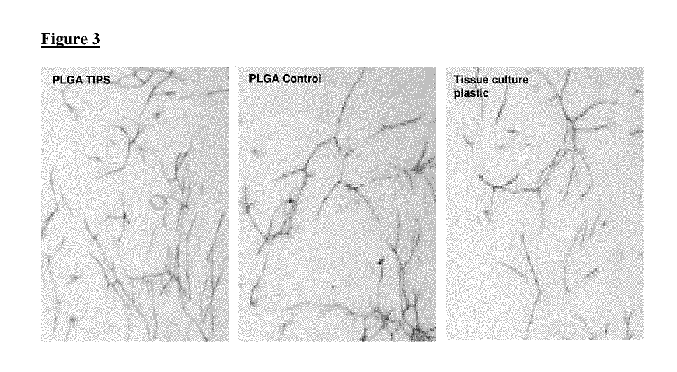

FIG. 3: The angiogenic activity of the supernatants collected from ADMSCs cultured on the different substrates was tested using an angiogenesis assay (V2a Kit; Cellworks).

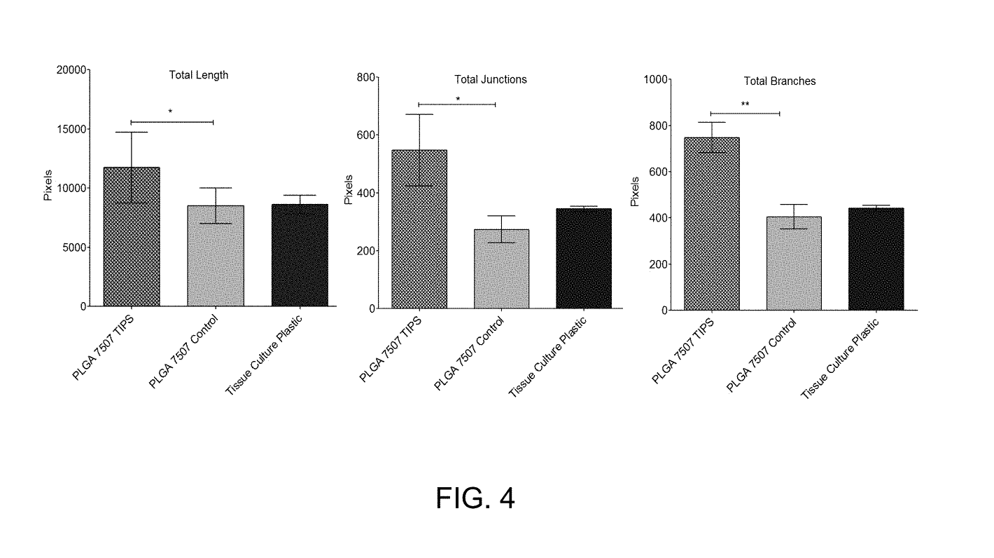

FIG. 4: Image analysis was used to quantify tubule length, number of junctions and tubule branches of the capillary-like vessels formed in the presence of supernatants collected over 14 days from cells cultured on the PLGA TIPS surfaces compared with the control groups. Tubule length, number of junctions and tubule branches were all significantly increased by supernatants collected from cells cultured on the PLGA TIPS surfaces compared with the control groups. (*p<0.05, **p<0.01).

### EXAMPLES

### Example 1

### Preparation of TIPS Coated Surfaces

Test surfaces intended for use as a substrate for cell culture were fabricated using the TIPS process. To achieve this, a biodegradable polymer (poly-DL-lactide-co-glycolide; PURASORB PDLG7507) dissolved in dimethyl carbonate at 10 wt % was prepared as a coating solution. 13 mm diameter borosilicate glass coverslips were dipped into the polymer solution then immediately plunged into liquid nitrogen. The excess liquid nitrogen was removed and the coated coverslips transferred to a −80° C. freezer. The coverslips were subsequently placed into a freeze drier and lyophilized for 18 hours until the solvent had been removed.

Control samples not exposed to the TIPS process were prepared by dipping 13 mm diameter borosilicate glass coverslips into the polymer solution and allowing the solvent to evaporate via air-drying for 48 hours.

The surface of the polymer coated coverslips was examined using a Jeol 7401-high resolution field emission scanning electron microscope at a magnification ×1000 (FIG. 1).

The surface topographies (See FIG. 1) prepared using the TIPS process provide a microenvironment with biophysical cues that result in cells secreting factors that result in increased angiogenesis (see FIGS. 2 to 4).

### Example 2

Secretion of VEGF from Human Adipose-Derived Mesenchymal Stem Cells

Human Adipose Derived Stem Cells (ADMSCs; StemPro® Human Adipose-Derived Stem Cells; ThermoFisher Scientific; isolated from human lipoaspirate tissue and cryopreserved from primary cultures) were grown on the TIPS polymer coated coverslips along with control polymer coverslips, and tissue culture plastic; n=4 per group. The coverslips were placed into a 24 well tissue culture plate and 1×10⁵ cells were added to each well in 1 mL MesenPRO RS™ Medium (ThermoFisher Scientific). The culture medium was collected at regular intervals (days 2, 4 and 7) and stored at −80° C. until further analysis. 1 ml of fresh MesenPRO RS™ Medium was added to the wells.

The quantity of cells attached to the different surfaces was measured at different time points so that the amount of angiogenic growth factor secreted per cell could be calculated.

After removal of the culture medium the number of cells was measured using the CyQUANT NF Cell proliferation assay (ThermoFisher) following the manufacturer's instructions. Each treatment group was analysed with a replicate of n=6. After incubation at 37° C. for 1 hour, 100 μl of the CyQUANT NF reaction was transferred into a 96 black walled well plate and the fluorescence intensity measured at 485 nm/535 nm. Background fluorescence intensity measured from negative control wells containing no cells was subtracted from the measurements collected for the test surfaces.

The quantity of vascular endothelial growth factor (VEGF) secreted by cells cultured on the different test substrates was measured using an ELISA (hVEGF ELISA DuoSet sandwich ELISA R&D Systems), following the manufacturer's instructions. Each treatment group was analysed with a replicate of n=6. The quantity of VEGF secreted per cell was calculated by dividing the amount of VEGF measured using the ELISA by the number of cells measured using the CyQUANT NF Cell proliferation assay (FIG. 2).

As is evident from FIG. 2, the TIPS surface resulted in increased secretion of VEGF.

### Example 3

### Angiogenic Secretome Activity

In addition to measuring the amount of VEGF secreted, the secretion of angiogenic growth factors (angiogenic secretome) from cells cultured on the different test substrates was measured using a commercially available angiogenesis assay (Va2 Kit: From Vasculogenesis to Angiogenesis; Cell Works Product) following the manufacturer's instructions (FIG. 3).

Human Adipose Derived Stem Cells (ADMSCs; StemPro® Human Adipose-Derived Stem Cells; ThermoFisher Scientific; isolated from human lipoaspirate tissue and cryopreserved from primary cultures) were grown on the TIPS polymer coated coverslips along with control polymer coverslips, and tissue culture plastic; n=4 per group. The coverslips were placed into a 24 well tissue culture plate and 1×10⁵ cells were added to each well in 1 mL MesenPRO RS™ Medium (ThermoFisher Scientific). The culture medium was collected at regular intervals (days 2, 4, 7, 9, 11, 14) and stored at −80° C. until further use. (The samples analysed correspond with the samples analysed in Example 2 above.)

Experimental groups included in the angiogenesis assay were: (i) Supernatant collected from ADMSCs grown on PLGA 7507 TIPS coverslips (n=4 wells); (ii) Supernatant collected from ADMSCs grown PLGA 7507 control coverslips (n=4 wells); (iii) Supernatant collected from ADMSCs grown tissue culture plastic (n=2 wells). Additional controls included in the experiment were VEGF, Suramin and medium only −n=2 wells each.

At days 2, 4, 7, 9, 11, 14 of the angiogenesis assay, medium from each well was removed and replaced with 1 ml of fresh culture medium containing 50% V2a Growth Medium and 50% conditioned medium collected from the cultures consisting of ADMSCs growing on the test surfaces at the corresponding time point (days 2, 4, 7, 9, 11, 14). Additional controls in the assay included V2a Growth Medium alone, VEGF control, Suramin control).

At the end of the incubation period, endothelial cells in the wells were stained for CD31 (PECAM-1) and imaged using photomicroscopy.

As can be seen from FIG. 3, the TIPS surface resulted in increased angiogenesis.

### Example 4

### Image Analysis of Endothelial Tubule Formation

Cellworks AngioSys 2.0 Image Analysis Software was used for semi-automated analysis of angiogenesis by measuring the number of endothelial tubules, the number of junctions, the total tubule length, and the mean tubule length for each image obtained in Example 3. The results obtained are presented Table 1 below and in FIG. 4.

TABLE 1

| | | | Tissue culture |
|---|---|---|---|
| | TIPS PLGA | Control PLGA | plastic |
| Tubule length | 11732 ± 1502 | 8504 ± 752 | 8607 ± 389 |
| | P < 0.05 | | |
| No. tubule | 547 ± 123 | 274 ± 47 | 339 ± 11 |
| junctions | P < 0.05 | | |
| No. tubule | 748 ± 66 | 405 ± 53 | 442 ± 14 |
| branches | P < 0.01 | | |

N = 4 replicates per group

The TIPS surface resulted in increased tubule length, more tubule junctions and more tubule branches.

All cited references are herein incorporated in their entirety.

1. (canceled)

2. A method for obtaining an angiogenic factor from one or more cells comprising:
i) ;
ii) attaching one or more cells which produce an angiogenic factor to a structure obtained by thermally induced phase separation;
iii) culturing the one or more attached cells in appropriate conditions for the cells to produce the angiogenic factor; and
iv) isolating the angiogenic factor produced by the cells.

3. The method of claim 2 wherein the structure is a coating on a substrate.

4. The method of claim 3 wherein the substrate is a cell culture plate or flask.

5. The method of claim 2 wherein the cells are Human Adipose Derived Stem Cells (ADMSCs).

6. The method of claim 2 wherein the angiogenic factor is Vascular Endothelial Growth Factor (VEGF).

7. The method of claim 2 wherein the isolating is by collection of the liquid in which the cells are cultured.

8. The method of claim 3 wherein the thermally induced phase separation comprises:
i) coating the substrate with a polymer and a solvent;
ii) quenching the substrate having the polymer and solvent coating in a quenching fluid; and
iii) freeze-drying the coating to obtain the substrate coated with the structure.

9. The method of claim 8 wherein the polymer is PLGA.

10. The method of claim 8 wherein the solvent is dimethyl carbonate.

11. The method of claim 8 wherein the quenching fluid is liquid nitrogen.

12. The method of 2 wherein the method is performed in vitro.

13. (canceled)

14. A method for obtaining Vascular Endothelial Growth Factor (VEGF) from one or more Adipose Derived Stem Cells (ADMSCs) comprising:
i) attaching one or more ADMSCs to a coating on a tissue culture plate or flask formed by thermally induced phase separation;
ii) culturing the ADMSCs in appropriate conditions for the cells to produce VEGF; and
iii) isolating the VEGF produced by the cells.

15. A method of treating cardiovascular disease comprising:
administering to a human with cardiovascular disease a structure produced by thermally induced phase separation, wherein the structure has one or more cells which produce an angiogenic factor attached to its surface.

16. The method according to claim 15 wherein the cardiovascular disease is Peripheral Artery Disease (PAD).

17. The method according to claim 15 wherein the structure is a microsphere.

18. The method according to claim 15 wherein the structure is a coating on a substrate.

19. The method according to claim 18 wherein the substrate is a medical device.

20. The method according to claim 19 wherein the medical device is a stent.

21. The method according to claim 15 wherein the cells are ADMSCs.

22. The method according to claim 15 wherein the angiogenic factor is VEGF.

23. The method of claim 18 wherein the thermally induced phase separation comprises:
i) coating the substrate with a polymer and a solvent;
ii) quenching the substrate having the polymer and solvent coating in a quenching fluid; and
iii) freeze-drying the coating to obtain the substrate coated with the structure.

24. The method of claim 17 wherein the thermally induced phase separation comprises:
i) dissolving a polymer in a solvent to form a solution;
ii) quenching droplets of the solution in a quenching fluid; and
iii) freeze-drying the resultant spheres.

25. The method of claim 24 wherein the polymer is PLGA.

26. The method of claim 24 wherein the solvent is dimethyl carbonate.

27. The method of claim 24 wherein the quenching fluid is liquid nitrogen.

28.-41. (canceled)
